(19) United States
(12) Patent Application Publication
(10) Pub. No.: US 2026/0166043 A1
(43) Pub. Date: Jun. 18, 2026

(54) COMPOSITIONS AND FORMULATIONS FOR CREATING MENTAL AND PHYSICAL ENERGY

(71) Applicant: Korrect Life, LLC, Austin, TX (US)

(72) Inventor: Eric Guerrieri, Bastrop, TX (US)

(21) Appl. No.: 19/407,917

(22) Filed: Dec. 3, 2025

Related U.S. Application Data
(60) Provisional application No. 63/733,740, filed on Dec. 13, 2024.

(51) Int. Cl. A61K 31/522; A61K 31/352; A61K 36/22; A61K 36/74; A61K 36/82; A61K 36/9062; A61P 25/26

(52) U.S. Cl. CPC A61K 31/522; A61K 31/352; A61K 36/22; A61K 36/742; A61K 36/82; A61K 36/9062; A61P 25/26

(57) ABSTRACT

Provided herein are methods and compositions for creating mental and physical energy without caffeine. The formulation and method comprises an effective amount of blend of at least three ingredients, paraxanthine, Mangifera indica, and Alpinia galanga which are included in one formulation embodiment.

### CROSS-REFERENCE TO RELATED APPLICATIONS

The present application claims priority to U.S. provisional application Ser. No. 63/733,740 filed Dec. 13, 2024, herein incorporated by reference in its entirety.

### BACKGROUND

This invention relates to a combination of ingredients for increasing mental and physical energy in a subject.

For centuries, humans have relied on caffeine for increasing mental and physical energy. However, caffeine has negative side effects. Therefore, there is a need to provide sustained mental and/or physical energy without the use of caffeine.

The present invention attempts to solve these problems, as well as others.

### SUMMARY OF THE INVENTION

Provided herein are methods and compositions for creating mental and physical energy.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

The term “synergistic” as used herein refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-anxiety; ingredient B is pro-anxiety; when A and B are combined, said combination produces an anti-anxiety effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The term “formulation” as used herein refers to a composition which is pharmaceutically acceptable and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The formulation includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes).). Exemplary prodrugs release the corresponding free acid upon cleavage, and the hydrolyzable ester-forming residues of the compounds of the present invention.

In the present disclosure, an “effective amount” or an “effective dose” of paraxanthine, Mangifera indica, and Alpinia galanga, or composition refers to an amount of paraxanthine, Mangifera indica, and Alpinia galanga that, once administered to a subject, will reach the subject's bloodstream, intestine, and/or bodily tissues. The term “effective amount” as used herein is intended to mean the amount of the composition administered to increase mental energy and/or physical energy. The effective amounts of the agent in the composition of the invention are effective over a wide dosage range and are generally administered in a pharmaceutically effective amount. It will be understood, however, that the effective amount of the compound actually administered will be determined in the light of the relevant circumstances, including the condition to be treated, the chosen route of administration, the age, weight, and response of the individual patient, the severity of the patient's symptoms, and the like.

### DESCRIPTION OF THE EMBODIMENTS

Generally speaking, the composition or formulation comprises an effective amount of blend of at least three ingredients, paraxanthine, Mangifera indica, and Alpinia galanga which are included in one formulation embodiment. In another embodiment, the composition or formulation comprises an effective amount of paraxanthine, Mangifera indica, and Alpinia galanga with decaf green tea, decaf green coffee bean, or quercetin in other embodiments. Compositions of the disclosure can be administered as a therapeutic and/or a supplement for creating mental and/or physical energy. Treatments designed to create mental and/or physical energy can result in an effect of the dopamine pathway. Compositions and methods are provided for increasing energy, mental focus, improved endurance, increased reaction time, prevents mental fatigue, increase dopamine, inhibit Catechol-O-methyltransferase (COMT).

Alpinia galanga is in the ginger family and the rhizome is used. The leaf portion of this plant can be used in the context of the present invention (at the exclusion of other portions of the plant). The extract can be an aqueous extract, a glycolic extract, an alcoholic extract, or any combination thereof. Alpinia galanga rhizome contains the flavonol galangin. The rhizome contains an oil known as galangol, which upon fractional distillation produces cineol (which has medicinal properties), pinene, and eugenol, among others. Alpinia galanga phytoconstituents interact with neurotransmitters such as dopamine and acetylcholinesterase that play a role in alertness and focus. Galangin, a flavonol derived from rhizoma alpiniae Officinarum, inhibits acetylcholinesterase activity in vitro.

Paraxanthine is a central nervous stimulant and the main metabolite of caffeine in humans. It's a member of the xanthine family of alkaloids, which also includes caffeine, theobromine, and theophylline. Paraxanthine is formed in the liver through cytochrome P450 1A2 N-demethylation of caffeine. It's excreted in urine along with other metabolites. Paraxanthine, also known as 1,7-dimethylxanthine, is a metabolite of theophylline and theobromine, two well-known stimulants found in coffee, tea, and chocolate mainly in the form of caffeine.

The systemic administration of paraxanthine profoundly affects hemodynamic parameters, such as blood pressure and heart rate, and stimulates the respiratory and central nervous systems. Paraxanthine exert their effects by at least five primary mechanisms: 1) the nonselective antagonism of ARs (all subtypes), 2) the competitive inhibition of phosphodiesterase (PDE isoenzymes III and IV), 3) the modulation of GABAA receptors, 4) the mobilization of intracellular calcium, and 5) the activation of histone deacetylase (isoenzyme type II). However, due to the very similar affinities of caffeine and adenosine to binding A1 and A2A receptors, paraxanthine has been shown that after the moderate consumption of paraxanthine, their effects are primarily due to the antagonism of ARs.

Paraxanthine acts as an antagonist of adenosine receptors (ARs), meaning it blocks the activity of adenosine by binding to its receptors, which is the primary mechanism behind its stimulant effects; essentially, by blocking adenosine's ability to induce sleepiness, paraxanthine promotes wakefulness and increased activity levels. Paraxanthine can antagonize both A1 and A2A adenosine receptors.

The structural formulation of paraxanthine is shown below in formula (1):

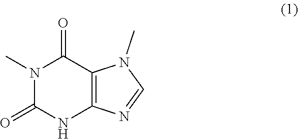

Several derivatives of paraxanthine have been synthesized to improve bioactivity and bioavailability.

Mangifera indica (M. indica), commonly known as mango, is an evergreen species of flowering plant in the family Anacardiaceae. Mangiferin (a pharmacologically active hydroxylated xanthone C-glycoside) is extracted from mango at high concentrations from the young leaves (172 g/kg), bark (107 g/kg), and from old leaves (94 g/kg). A large variety of chemical compounds have been reported in M. indica. Among these, polyphenols (flavonoids, xanthones, and phenolic acids) are the most abundant compound types in M. indica. Mangiferin, gallic acid, catechins, quercetin, kaempferol, protocatechuic acid, ellagic acids, propyl and methyl gallate, rhamnetin, and anthocyanins are the major polyphenolic compounds found in M. indica.

Cognitive enhancing effects and improvement in memory impairment by M. indica fruit pulp extract. M. indica fruit pulp extract show increased cholinergic neurons density and decreased oxidative stress in rates, which illustrates possible cognitive enhancing effects of M. indica fruit pulp. Neuroprotective activities of methanol and aqueous extracts of M. indica leaf showed that methanol extract and aqueous extract can effectively protect H2O2-induced neuroblastoma cells from oxidative damage. M. indica bark can exert hepatoprotective effects to regulates metabolism and detoxification of toxic substances in the liver.

There are a number of disadvantages with taking three supplements for a single effect. One difficulty relates to convenience. Taking more supplements in a day requires more effort. Maintaining adequate supplies of supplements becomes more difficult when more supplements are being taken, particularly when the number of doses in a package is different for the different supplements. Taking the correct amount of each supplement is progressively more difficult with an increasing number of supplements taken. It is easier to make a mistake with the quantity taken and easier to miss taking the supplement. Any synergistic effects from taking three supplements at the same time can be more difficult to achieve than taking a single supplement if mixing in the stomach is required to achieve the desired synergy. Any synergistic effects of three supplements may also be more strongly impacted by the presence and type of food than a single supplement. The form of the supplement can also be a factor. When the paraxanthine, Mangifera indica, and Alpinia galanga are taken in a single form, accurate dosing is achieved to increase mental and physical energy.

In one embodiment, the method comprises administering or taking paraxanthine, Mangifera indica, and Alpinia galanga in a single formulation to increase synergy between the three supplements. The method comprises combining paraxanthine, Mangifera indica, and Alpinia galanga into a formulation to increase the mental energy and/or physical energy via the dopamine pathway.

In one embodiment, the method of manufacturing comprises combining paraxanthine, Mangifera indica, and Alpinia galanga into a formulation. In another embodiment, the method of manufacturing comprises providing a ratio of paraxanthine, Mangifera indica, and Alpinia galanga.

This invention overcomes the above disadvantages by combining paraxanthine, Mangifera indica, and Alpinia galanga into a single composition to increase energy, mental focus, improve endurance, increase reaction time, prevent mental fatigue, increase dopamine, or inhibit COMT. This invention also helps to maximize the synergy of taking the three supplements together combined with additional ingredients detailed below.

Decaffeinated green tea is a type of tea that is made from Camellia sinensis leaves and buds that have not undergone the same withering and oxidation process. Decaffeinated green tea includes theobromine, and theophylline, which give it stimulant effects. Polyphenols found in green tea include epigallocatechin gallate (EGCG), epicatechin gallate, epicatechins and flavanols. Other components include three kinds of flavonoids, known as kaempferol, quercetin, and myricetin. Although the mean content of flavonoids and catechins in a cup of green tea is higher than that in the same volume of other food and drink items that are traditionally considered to promote health, flavonoids and catechins have a biological effect in human. Catechins are flavonoids and have antioxidant properties. Green tea extract rich in the polyphenol catechin epigallocatechin gallate has been shown to increase sympathetic nervous system activity, and to increase 24-h energy expenditure and fat oxidation.

Green tea extract can increase thermogenesis by 35-43%, which could contribute to a significant increase in total 24-hour energy expenditure. Mental performance: Green tea can improve mental arithmetic task performance. Green tea can help reduce feelings of fatigue and promote recovery.

In one embodiment, paraxanthine, Mangifera indica, and Alpinia galanga combines with decaffeinated green tea to increase energy, mental focus, improve endurance, increase reaction time, prevent mental fatigue, increase dopamine, or inhibit COMT. This paraxanthine, Mangifera indica, and Alpinia galanga composition supplements with decaffeinated green tea in a single composition form makes it synergistically increase mental and physical energy in humans via the dopamine pathway. Since the supplements are combined into a single composition, this method of combining the supplements minimizes the mass/bulk of the single dose form. In addition, the ratio of the supplements is fixed at the molecular level, eliminating variability that can occur when blending the different compounds. Decaffeinated Green Coffee Bean Extract (GCBE) has several health benefits including increasing energy and weight management. The physiological functions of GCBE are mainly attributed to the chlorogenic acids (CGAs) present as a major phytoconstituent. Other bioactive constituents include vanillic, p-coumaric and feruloyl acids, trigonelline, tannins and anthraquinones. CGA-7 is a standardized extract from C. arabica green coffee beans (NLT 50% CGAs) containing seven isomers of CGAs, 5-caffeoylquinic acid being the major constituent.

GCBEs regulate blood sugar levels and stimulate metabolism. Additionally, emerging research hints at the neuroprotective properties of GCBEs, suggesting potential support for cognitive function. GCBE attenuated oxidative damage (increased cortical reduced glutathione and superoxide dismutase activity), while decreased malondialdehyde. It retarded the activation of acetylcholine esterase, increased acetylcholine level. GCBE improving cholinergic activity, learning, and memory ability.

In one embodiment, paraxanthine, Mangifera indica, and Alpinia galanga combines with GCBE to increase energy, mental focus, improve endurance, increase reaction time, prevent mental fatigue, increase dopamine, or inhibit COMT. This paraxanthine, Mangifera indica, and Alpinia galanga composition supplements with GCBE in a single composition form makes it synergistically increase mental and physical energy in humans via the dopamine pathway. Since the supplements are combined into a single composition, this method of combining the supplements minimizes the mass/bulk of the single dose form. In addition, the ratio of the supplements is fixed at the molecular level, eliminating variability that can occur when blending the different compounds.

Quercetin is a plant flavonol from the flavonoid group of polyphenols. It is found in many fruits, vegetables, leaves, seeds, and grains; capers, red onions, and kale are common foods containing appreciable amounts of it. It has a bitter flavor and is used as an ingredient in dietary supplements, beverages, and foods. Quercetin's UPAC name is 3,3′,4′,5,7-Pentahydroxyflavone. Quercetin's Systematic IUPAC name is 2-(3,4-Dihydroxyphenyl)-3,5,7-trihydroxy-4H-1-benzopyran-4-one. Quercetin's other names include 5,7,3′,4′-flavon-3-ol, Sophoretin, Meletin, Quercetine, Xanthaurine, Quercetol, Quercitin, Quertine, Flavin meletin.

In one embodiment, paraxanthine, Mangifera indica, and Alpinia galanga combines with Quercetin to increase energy, mental focus, improve endurance, increase reaction time, prevent mental fatigue, increase dopamine, or inhibit COMT. This paraxanthine, Mangifera indica, and Alpinia galanga composition supplements with Quercetin in a single composition form makes it synergistically increase mental and physical energy in humans via the dopamine pathway. Since the supplements are combined into a single composition, this method of combining the supplements minimizes the mass/bulk of the single dose form. In addition, the ratio of the supplements is fixed at the molecular level, eliminating variability that can occur when blending the different compounds.

In one embodiment the paraxanthine, Mangifera indica, and Alpinia galanga combines with decaffeinated green tea and decaffeinated green coffee bean, and quercetin to increase energy, mental focus, improve endurance, increase reaction time, prevent mental fatigue, increase dopamine, or inhibit COMT.

In one embodiment the paraxanthine, Mangifera indica, and Alpinia galanga combines with decaffeinated green tea and decaffeinated green coffee bean to increase energy, mental focus, improve endurance, increase reaction time, prevent mental fatigue, increase dopamine, or inhibit COMT.

In one embodiment the paraxanthine, Mangifera indica, and Alpinia galanga combines with decaffeinated green tea and quercetin to increase energy, mental focus, improve endurance, increase reaction time, prevent mental fatigue, increase dopamine, or inhibit COMT.

In one embodiment the paraxanthine, Mangifera indica, and Alpinia galanga combines with decaffeinated green coffee bean and quercetin to increase energy, mental focus, improve endurance, increase reaction time, prevent mental fatigue, increase dopamine, or inhibit COMT.

In one embodiment the paraxanthine, Mangifera indica, and Alpinia galanga combines with decaffeinated green tea and decaffeinated green coffee bean, and quercetin to increase energy, mental focus, improve endurance, increase reaction time, prevent mental fatigue, increase dopamine, or inhibit COMT.

In some cases, the paraxanthine, Mangifera indica, and Alpinia galanga combines with decaffeinated green tea and decaffeinated green coffee bean, and quercetin composition may result in a favorable change in one or more indicators associated or correlated with mental health or physical energy, including changes in biomarkers, metabolites, physiology, pathways, or indicators associated with the condition.

### Dosage Form

The paraxanthine, Mangifera indica, and Alpinia galanga formulation can be compressed into tablet form or encapsulated. The paraxanthine, Mangifera indica, and Alpinia galanga formulation can also be included in a blend of other supplements. Other dosage forms can include gummy formulations or powder forms that can be dissolved in a liquid.

In some embodiments, a paraxanthine, Mangifera indica, and Alpinia galanga formulation provided herein may comprise between about 50 mg to about 1000 mg of paraxanthine per effective dose, alternatively 50 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or between 200 mg-400 mg of paraxanthine per effective dose.

In some embodiments, a paraxanthine, Mangifera indica, and Alpinia galanga formulation provided herein may include about 1 mg to 500 mg of Mangifera indica per effective dose. For example, a paraxanthine, Mangifera indica, and Alpinia galanga formulation provided herein may comprise 1 mg to 1000 mg, 20 mg to 480 mg, 30 mg to 470 mg, 40 mg to 460 mg, 40 mg to 450 mg, 30 mg to 450 mg, 20 mg to 450 mg, 10 mg to 450 mg, 10 mg to 500 mg, 20 mg to 550 mg, 30 mg to 550 mg, 35 mg to 550 mg, 40 mg to 550 mg, 40 mg to 555 mg, 35 mg to 555 mg, 35 mg to 555 mg, 35 mg to 550 mg, 35 mg to 545 mg, 40 mg to 545 mg, 45 mg to 575 mg, 45 mg to 555 mg, 45 mg to 555 mg, 45 mg to 565 mg, 45 mg to 555 mg, 45 mg to 250 mg, 55 mg to 555 mg, 55 mg to 550 mg, or between 55 mg to 550 mg of Mangifera indica per effective dose. Alternatively, the dose may comprise 50 mg to about 500 mg of Mangifera indica per effective dose.

In some embodiments, a paraxanthine, Mangifera indica, and Alpinia galanga formulation provided herein may include about 300 mg to 600 mg of Alpinia galanga per effective dose. For example, a paraxanthine, Mangifera indica, and Alpinia galanga formulation provided herein may comprise 250 mg to 675 mg, 250 mg to 650 mg, 250 mg to 575 mg, 250 mg to 600 mg, 275 mg to 700 mg, 275 mg to 575 mg, 275 mg to 550 mg, 275 mg to 625 mg, 275 mg to 600 mg, 300 mg to 900 mg, 100 mg to 275 mg, 100 mg to 250 mg, 100 mg to 225 mg, 125 mg to 250 mg, 125 mg to 375 mg, 125 mg to 250 mg, 150 mg to 250 mg, 150 mg to 275 m mg g, or between 180 mg to 250 mg of Alpinia galanga per effective dose. Alternatively, the dose may comprise about 250 mg of Alpinia galanga per effective dose.

In some embodiments, a paraxanthine, Mangifera indica, Alpinia galanga and decaffeinated green tea formulation provided herein may include about 60 mg to 300 mg of decaffeinated green tea per effective dose. For example, a paraxanthine, Mangifera indica, Alpinia galanga and decaffeinated green tea formulation provided herein may comprise 50 mg to 400 mg, 50 mg to 350 mg, 50 mg to 375 mg, 50 mg to 400 mg, 55 mg to 300 mg, 55 mg to 275 mg, 55 mg to 350 mg, 55 mg to 325 mg, 65 mg to 300 mg, 70 mg to 300 mg, 70 mg to 375 mg, 70 mg to 350 mg, 70 mg to 325 mg, 65 mg to 350 mg, 65 mg to 375 mg, 65 mg to 350 mg, 65 mg to 250 mg, 60 mg to 375 mg, or between 10 mg to 725 mg of decaffeinated green tea per effective dose. Alternatively, the dose may comprise about 55 mg to 325 mg of decaffeinated green tea per effective dose.

In some embodiments, a paraxanthine, Mangifera indica, Alpinia galanga and decaffeinated green coffee bean formulation provided herein may include about 50 mg to 700 mg of decaffeinated green coffee bean per effective dose. For example, a paraxanthine, Mangifera indica, Alpinia galanga and decaffeinated green coffee bean formulation provided herein may comprise 70 mg to 660 mg, 70 mg to 650 mg, 70 mg to 675 mg, 70 mg to 550 mg, 70 mg to 575 mg, 75 mg to 575 mg, 75 mg to 650 mg, 75 mg to 700 mg, 75 mg to 625 mg, 80 mg to 700 mg, 80 mg to 675 mg, 80 mg to 650 mg, 80 mg to 625 mg, 85 mg to 650 mg, 85 mg to 675 mg, 85 mg to 700 mg, 90 mg to 650 mg, 90 mg to 675 mg, or between 90 mg to 700 mg of decaffeinated green coffee bean per effective dose. Alternatively, the dose may comprise about 100 mg to 600 mg of decaffeinated green coffee bean per effective dose.

In some embodiments, a paraxanthine, Mangifera indica, Alpinia galanga and Quercetin formulation provided herein may include about 10 mg to 500 mg of Quercetin per effective dose. For example, a paraxanthine, Mangifera indica, Alpinia galanga and Quercetin formulation provided herein may comprise 30 mg to 400 mg, 30 mg to 450 mg, 30 mg to 475 mg, 30 mg to 425 mg, 45 mg to 400 mg, 45 mg to 375 mg, 45 mg to 350 mg, 45 mg to 325 mg, 45 mg to 300 mg, 70 mg to 500 mg, 70 mg to 475 mg, 70 mg to 450 mg, 70 mg to 425 mg, 65 mg to 425 mg, 65 mg to 475 mg, 65 mg to 450 mg, 55 mg to 450 mg, 55 mg to 425 mg, or between 55 mg to 500 mg of Quercetin per effective dose. Alternatively, the dose may comprise about 40 mg to 300 mg of Quercetin per effective dose.

In one embodiment, the formulation is blended with other nootropic supplements such as phosphatidylserine, L-theanine, creatine monohydrate, and/or an appropriate amount of another nootropic compound.

In one embodiment, the paraxanthine, Mangifera indica, Alpinia galanga formulation is in the form of capsules that consumers can take orally. The paraxanthine, Mangifera indica, Alpinia galanga formulation is free from any contaminants, including foreign material, pesticides, bacterial pathogens, molds, residual solvents, heavy metals. A certificate of analysis (COA) documented the analytical tests for paraxanthine, Mangifera indica, Alpinia galanga formulation.

The methods described herein may comprise administering daily, or every other day, or once a week, an effective dose of the paraxanthine, Mangifera indica, Alpinia galanga formulation. In an embodiment, the paraxanthine, Mangifera indica, Alpinia galanga formulation is administered daily for 1-2 weeks, 1-3 weeks, 1-12 week, 4-8 weeks, 8-12 weeks, 1-12 weeks, 4-12 weeks, 8-12 weeks, or 12-15 weeks, or more, or for another period of time according to the present invention.

The paraxanthine, Mangifera indica, Alpinia galanga formulation of the present invention may be administered in combination with one or more nutraceutically acceptable carriers and/or other excipients. The active ingredients in such formulations may comprise from 1% by weight to 99% by weight, or alternatively, 0.1% by weight to 99.9% by weight. “Nutraceutically acceptable carriers and/or other excipients” means any carrier, diluent or excipient that is compatible with the other ingredients of the paraxanthine, Mangifera indica, Alpinia galanga formulation and not deleterious to the user. In accordance with one embodiment, suitable nutraceutically acceptable carriers can include oils, plant-based oils, Medium-chain triglyceride (MCT) oil, coconut oil, palm kernel oil, Hemp seed oil, Olive oil, Avocado oil, and combinations thereof. Carriers may include dicalcium phosphate, rice flour, maltodextrin, microcrystalline cellulose, etc. Other excipients may include binders (such as hydroxypropyl cellulose, Hypromellose, various gums, etc.), flow agents/anticaking agents (such as silicon dioxide, etc.), disintegrants (such as croscarmellose sodium, etc.), lubricants/mold release agents (such as magnesium stearate, calcium laurate, etc.), flavorings/sweeteners, coloring, or other excipients as needed for the dosage form.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, gummy, chocolate or cookie. In certain embodiments, the tablet or pellets are an immediate release or slow or controlled release dosage forms. In certain embodiments, the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the paraxanthine, Mangifera indica, Alpinia galanga formulation at the intestine.

In certain embodiments, the paraxanthine, Mangifera indica, Alpinia galanga formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form. Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Preferred dosage forms include, but are not limited to, any liquid or semi solid or solid dosage form. The paraxanthine, Mangifera indica, Alpinia galanga formulation may be formulated in a medicament by preparing a topical or mucosal or oral delivery system. The topical delivery system may be in the form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the source of paraxanthine, Mangifera indica, Alpinia galanga formulation further comprises one or more excipients selected from the group consisting of solvents, stabilizers, suspending agents, emulsifiers, release modifying, targeting and viscosity agents and combinations thereof.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Effect of Paraxanthine, Mangifera Indica, Alpinia Galanga on Mental Alertness and Sustained Attention without Caffeine

Objective: Although Alpinia galanga has been reported to improve cognitive performance in animals, paraxanthine, Mangifera indica, Alpinia galanga has not been thoroughly studied for its potential psychostimulant effect in humans. A randomized, double-dummy, double-blind, placebo-controlled cross-over study will be conducted to determine the effect of paraxanthine, Mangifera indica, Alpinia galanga on mental alertness and sustained attention in comparison with caffeine and placebo in participants with a habitual caffeine intake.

Methods: Fifty-nine participants (18-40 years and body mass index of ≥18.5 and <25.00 kg/m2) with moderate caffeine consumption were enrolled. The participants had a Generalized Anxiety Disorder-7 score ≤7, Patient Health Questionnaire-9 score ≤14 and a Jin Fan's Attention Network Test alertness score of 50±20 ms. The interventional product (placebo, paraxanthine, Mangifera indica, Alpinia galanga, and caffeine) will be administered to the participants, followed by sequential administration of the remaining interventions on the consecutive study visits; the effects on mental alertness, sustained attention, and sleep architecture, along with safety and tolerability, will be analyzed by validated methods.

Possible Results: In the paraxanthine, Mangifera indica, Alpinia galanga group, the alertness score may be increased from baseline at 1 and 5 hours, respectively, indicating its efficacy to enhance mental alertness and the increase in alertness score as compared to placebo. In the composite group (with caffeine), mean response time may be significantly reduced at 3 hours.

Conclusions: paraxanthine, Mangifera indica, Alpinia galanga may induce a beneficial effect in mental alertness and compared with caffeine.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A composition for increasing mental and/or physical energy comprising: an effective amount of blend of at least three ingredients, paraxanthine, Mangifera indica, and Alpinia galanga.

2. The composition of claim 1, further comprising effective amount of decaffeinated green tea, decaffeinated green coffee bean, and quercetin.

3. The composition of claim 1, further comprising effective amount of decaffeinated green tea.

4. The composition of claim 1, further comprising effective amount of decaffeinated green coffee bean.

5. The composition of claim 1, further comprising effective amount of quercetin.

6. A method for increasing mental and/or physical energy in a subject, comprising: administering an effective amount at least three ingredients, paraxanthine, Mangifera indica, and Alpinia galanga to a subject; and increasing mental or physical energy in the subject.

7. The method of claim 6, further comprising administering an effective amount of decaffeinated green tea, decaffeinated green coffee bean, and quercetin.

8. The method of claim 6, further comprising administering an effective amount of decaffeinated green tea.

9. The method of claim 6, further comprising administering an effective amount of decaffeinated green coffee bean.

10. The method of claim 6, further comprising administering an effective amount of quercetin.
